(19) United States
(12) Patent Application Publication
(10) Pub. No.: US 2025/0176858 A1
(43) Pub. Date: Jun. 5, 2025

(54) METHOD FOR ASSESSING PAIN EXPERIENCED BY A PATIENT

(71) Applicant: BIOTRONIK SE & Co. KG, Berlin (DE)

(72) Inventors: Min Qu, Portland, OR (US); Pamela Shamsie Victoria Riahi, Portland, OR (US); Andrew B. Kibler, Lake Oswego, OR (US); Sean Slee, Tigard, OR (US)

(21) Appl. No.: 18/875,938

(22) PCT Filed: Jun. 15, 2023
(86) PCT No.: PCT/EP2023/066077

§ 371 Date: Dec. 17, 2024

Related U.S. Application Data
(60) Provisional application No. 63/354,816, filed on Jun. 23, 2022.

(30) Foreign Application Priority Data
Sep. 16, 2022 (EP) 22196009.9

(51) Int. Cl. A61B 5/11; A61B 5/00; A61B 5/07; G16H 40/67; G16H 50/20

(52) U.S. Cl. CPC A61B 5/1118; A61B 5/076; A61B 5/4824; A61B 5/7267; A61B 5/7275; G16H 40/67; G16H 50/20

(57) ABSTRACT

A method for assessing pain experienced by a patient (3) comprises: receiving a sensor signal (8) which has been generated in a sequence of epochs (10) by a sensor (2) configured for measuring a physical activity of the patient (3), wherein each epoch (10) comprises a sequence of measurement periods (11), wherein the sensor signal (8) has been generated in each measurement period (11); determining a signal intensity value (12) for each measurement period (11) from the sensor signal (8); classifying the signal intensity values (12) of each epoch (10) with different physical activity classes (13a, 13b, 13c, 13d) to obtain a classification result (15) for the epoch (10); and determining a behavior assessment score (16) indicative of the pain experienced by the patient (3) from the classification results (15) of different epochs (10).

The present invention relates to a computer-implemented method for assessing pain experienced by a patient. Furthermore, the invention relates to a data processing device, a medical system and a computer program for carrying out the method as well as to a computer-readable medium on which the computer program is stored.

The comprehensive assessment of pain and characterization of its impact on patients' quality of life is critical in pain management, including spinal cord stimulation therapy. Current pain assessments are usually self-reported and thus subjective.

As a subjective and complicated perception, the intensity of pain is usually evaluated in clinical settings by self-reported scales such as the Numeric Rating Scale (NRS) and quality of life questionnaires, for example the Oswestry Disability Index (ODI). Also, patient postures, including lying down, standing, sitting, and lying on one's side, can be tracked. Frequent changes in posture can be a sign of discomfort.

Although considered as “golden standard”, the accuracy of the self-reported NRS and ODI scales can be limited due to the variation of pain tolerance between individuals, psychological and environmental factors (such as pain catastrophizing) and communication bias with different medical care providers. Therefore, self-reported scale alone may be not sufficient to accurately estimate the intensity of pain and the level of global quality of life.

In addition, due to the variations in the posture-changing habit and response to pain, the detection of discomfort based on the posture changes may require tracking the posture patterns of each patient over a relatively long period of time as a baseline, which may make this method complex and less practical.

What is desired is an activity-derived metric which correlates to a patient's real-world quality of life and pain score.

It therefore may be seen as an objective of the present invention to provide an improved method for assessing pain experienced by a patient. Another objective of the invention may be to provide a device, a system, a computer program and a computer-readable medium for carrying out the method.

These objectives may be achieved by the subject-matter of the independent claims. Advantageous embodiments are defined in the dependent claims as well as in the corresponding specification and figures.

A first aspect of the invention relates to a computer-implemented method for assessing pain experienced by a patient. The method comprises at least the following steps: receiving a sensor signal which has been generated in a sequence of epochs by a sensor configured for measuring a physical activity of the patient, wherein each epoch comprises a sequence of measurement periods, wherein the sensor signal has been generated in each measurement period; determining a signal intensity value for each measurement period from the sensor signal; and classifying the signal intensity values of each epoch with different physical activity classes to obtain a classification result for the epoch.

The chronic pain population is known to be less active than the general population on average. During pain management using spinal cord stimulation, as the chronic pain of the patients is reduced, the patients are more likely to be able to move and exercise more.

According to an embodiment of the present invention, the method further comprises the step of determining a behavior assessment score indicative of the pain experienced by the patient from the classification results of different epochs.

Thus, as the Numeric Rating Scale is getting lower, which indicates that the pain level is reduced, the patients are exercising more and have higher activity signals. This correlation may be used to calculate pain scores automatically.

The main advantage of the method is that pain can be assessed based on an objective measurement from a sensor which is not affected by psychological factors or communication bias. Put simply, the sensor signal is divided into different intensity ranges to analyze, for example, different types of movement and/or different physical activities of the patient. The sensor signal, at different intensity and/or frequency ranges, can provide information as on the patient's posture change, movement ability, movement in specific period such as during sleep, and even respiration pattern and/or rate, which can be indicative of the change in pain severity and the impact of pain on the patient's life. For example, if patients stand up or walk more often during the day, or do more activities, this may indicate that they are experiencing an improvement in their quality of life. Thus, using the method as described above and below, changes in the patient's quality of life can be evaluated in a technically efficient and reliable manner.

The method may be carried out automatically by a processor.

The sensor may be part of a mobile device carried by the patient, e.g., a mobile medical device, smartphone, smartwatch, wearable (such as a fitness or sleep tracker), tablet or laptop. The sensor may be at least partially implanted in the patient's body, e.g., the patient's skin, heart, brain, spine, ear or blood vessel. Alternatively, the sensor may be a stationary sensor, e.g., part of a stationary medical device. For example, the sensor may be an accelerometer, gyroscope, altimeter, barometer, GPS receiver, electrical or optical heart sensor, pulse oximeter, camera, thermometer or a combination of at least two of these examples.

Accordingly, the sensor signal may, for example, indicate at least one of an acceleration, angular rate, position, orientation, velocity, heart rate, breathing rate, electrocardiogram, blood oxygen saturation or blood glucose level with respect to the patient.

The sensor signal may be analog or digital. The sensor signal may be generated continuously or in a sequence of time steps in each measurement period.

Further parameters may additionally be taken into account and combined with the sensor signal to develop an even more accurate pain evaluation metric.

Each measurement period may be as long as one full cycle of physical activity or longer. For example, each measurement period may be at least 1 second, 10 seconds, 1 minute or 1 hour.

Each epoch may be significantly longer than one measurement period. More specifically, each epoch may be as long as the sum of all measurement periods of the epoch or longer. For example, each epoch may be at least 1 minute, 1 hour, 1 day, 1 week or 1 month.

The duration of each epoch and/or of each measurement period may be constant or variable.

The signal intensity value may indicate an amplitude of the sensor signal with respect to one measurement period. For example, the signal intensity value may be an integration value or a statistical value such as a mean or median.

The physical activity classes may, for example, indicate different levels of physical activity, different types of physical activity or different types of body movements. Different physical activity classes may have different or identical weights.

The behavior assessment score may be seen as a pain intensity level or a variation thereof. The behavior assessment score may, for example, be a specific value or a specific value range selected from a predefined behavior assessment score range, e.g., between 1 and 10, wherein “1” is the lowest pain intensity level and “10” the highest pain intensity level. Other behavior assessment score ranges are also possible.

The behavior assessment score may be determined from the classification results of at least two different (e.g., consecutive) epochs, i.e., a current epoch and at least one previous epoch. The classification results may relate to the same patient or different patients.

For example, the classification results of a first epoch in the sequence of epochs, possibly in conjunction with a corresponding behavior assessment score or corresponding behavior assessment score range, may be used as a baseline or reference. These results may be derived with respect to the same patient, a different patient or a group of patients.

A second aspect of the invention relates to a data processing device comprising a processor configured for carrying out the method as described above and below. The data processing device may include hardware and/or software modules. In addition to the processor, the data processing device may include a memory and data communication interfaces for data communication with peripheral devices. For example, the data processing device may be part of a medical device (such as an implant or a remote for controlling an implant), smartphone, smartwatch, wearable (such as a fitness or sleep tracker), tablet, laptop, PC or server.

A third aspect of the invention relates to a medical system that comprises at least one sensor configured for measuring a physical activity of a patient and at least one data processing device as described above and below. The medical system may, for example, be a neurostimulator such as a spinal cord stimulator (other types of medical systems are possible as well). In this case, the medical system may comprise an implant which is implantable in the patient's body and a mobile device for controlling the implant remotely, such as, e.g., a remote, smartphone, smartwatch or tablet. In addition, the medical system may comprise a (remote) server for storing and/or processing data, e.g., pain-related data, provided by the implant and/or the mobile device. For example, the implant, the mobile device and the server may be interconnected for data communication. The medical system may be used not only to assess pain automatically or to complement conventional pain assessment methods (which are essentially based on the patient's subjective feedback), but also to automatically treat the patient in dependence of the (automatically obtained) pain assessment results in such a way that pain is alleviated accordingly.

According to an embodiment, the sensor signal is transmitted from the implant to the mobile device and/or from the implant directly to the server and/or from the implant via the mobile device to the server. Accordingly, the behavior assessment score may be determined from the sensor signal by the mobile device and/or by the server.

Further aspects of the invention relate to a computer program comprising instructions which, when the program is executed by a processor, cause the processor to carry out the method as described above and below and to a computer-readable medium in which the computer program is stored. The computer program may be executed by a processor of the data processing device. The computer-readable medium may be a volatile or non-volatile data storage device. For example, the computer-readable medium may be a hard drive, USB (universal serial bus) storage device, RAM (random-access memory), ROM (read-only memory), EPROM (erasable programmable read-only memory) or flash memory. The computer-readable medium may also be a data communication network for downloading program code, such as the Internet or a data cloud.

It has to be noted that features of the method as described above and below may be features of the data processing device, the computer program and the computer-readable medium, and vice versa.

Embodiments of the invention may be considered, without limiting the invention, as being based on the ideas and findings described below.

According to an embodiment, the behavior assessment score may be determined by further analyzing the sensor signal, e.g., its amplitude and/or frequency, in dependence of the classification results of different epochs. For example, the sensor signal may be further analyzed to detect, with respect to the determined physical activity classes, specific types of body movements which may indicate a change in the patient's quality of life, such as, for example, (pain-related) posture changes or respiration movements. This can help to improve the accuracy of the method.

According to an embodiment, classifying the signal intensity values may comprise: comparing each signal intensity value to different value ranges, each value range corresponding to one of the physical activity classes; when a value range which includes the signal intensity value is found: assigning the physical activity class of the found value range to the signal intensity value (and, thus, to the respective measurement period of the signal intensity value).

The value ranges may be predefined, i.e., static. For example, the value ranges may have been determined experimentally. Alternatively, the value ranges may be automatically or manually updated when the method is being carried out, e.g., based on the pain score, on a behavior assessment score trend over different epochs, on statistics of patient group assessment results, on significant changes in the sensor signal or on an input of the patient. According to an embodiment of the invention, the activity classes can be refined, and the ranges of different activity classes can be automatically updated based on activity signal trend and characteristics.

The value ranges may be seen as ranges of possible signal intensity values. Each value range may be defined by a lower limit and an upper limit (or threshold). Different value ranges may differ from each other in their lower limits, their upper limits or both their lower and upper limits. The lower limit and the upper limit of the same value range may differ from each other. It is, however, possible that the lower limit is equal to the upper limit of the same value range. In this case, the term “value range” may be interpreted as a single threshold value to which the signal intensity values may be compared.

Comparing each signal intensity value to different value ranges may, for example, mean that the signal intensity value is compared to both limits of each value range. If the signal intensity value is between the two limits or is equal to one of the limits of the value range, the signal intensity value will be assigned to the corresponding physical activity class.

Using different value ranges to classify the signal intensity values may simplify the implementation of the classification.

The accuracy of the method may be further improved by comparing the signal intensity values to at least three different value ranges, i.e., to at least three different subsets of thresholds.

Other classification methods may be used as well, such as, for example, statistical methods or methods based on machine learning.

According to an embodiment, the physical activity classes may include at least one of a first physical activity class comprising sedentary activities, a second physical activity class comprising light activities, a third physical activity class comprising moderate activities or a fourth physical activity class comprising vigorous activities. These kinds of physical activities are known to have a significant correlation with the patient's real-world quality of life and pain score.

For example, the sedentary activities may have a metabolic equivalent of task (MET) of 1.5 or lower than 1.5, the light activities a MET between 1.5 and 3.0, the moderate activities a MET between 3.0 and 6.0 and the vigorous activities a MET of 6.0 or higher than 6.0. Such MET ranges are known to have a significant correlation with the patient's real-world quality of life and pain score.

According to an embodiment, the classification result may indicate a physical activity duration with respect to each physical activity class. Accordingly, the behavior assessment score may be determined from the physical activity durations of different epochs.

According to an embodiment, the behavior assessment score can be a metric of a disability score, health score, behavior score, exercise score, or pain score. In particular, that metrics can be implemented as follows:
a pain score is e.g. similar to the Numeric Rate Scale (NRS), the Visual Analogue Scale (VAS), or any numeric or visual scale to rate pain. This score can be estimated by classifying activity signals analyzed over time into levels of pain or a continuous scale of pain. This can be done by, for example, creating a machine learning model and training it on data containing both reported pain scores and activity signals, so that the model learns how different levels/scores of pain correlate with different activity signals, and can then predict levels of pain based on new activity signals. The behavior assessment score can also be estimated with a script classifying/scoring activity levels over time into pre-defined pain classes/scores.
a disability score is e.g. by classifying activity signals analyzed over time into levels of disability or rating them on a continuous scale of disability. This can be done by, for example, creating a machine learning model and training it on data containing both filled disability questionnaires and activity signals, so that the model learns how different levels of disability correlate with different activity signals, and can then predict levels of disability based on new activity signals. The disability score can also be calculated with a script that identifies gait characteristics from the activity signals and classifies them into pre-defined disability classes or levels based on those signal signatures.
a behavior score is e.g. using machine learning methods similar to those described for the pain and disability scores, but with a behavior score as an outcome, which can correspond to different activity signal signatures over days/weeks/months.
an exercise score is e.g. average time spent exercising each day/week/month, activity intensity during exercise periods, or activity intensity patterns during exercise periods, or overall activity intensity over days/weeks/months, or a combination of exercise frequency and intensity over days/weeks/months, or any combination of exercise-related activity signal features. This can be estimated using machine learning methods similar to those described for the pain and disability scores.
a health score is e.g. a combination of one or all of the previously described metrics, or a general health score estimated using machine learning methods similar to those described for the pain and disability scores.

There may be a counter which counts how many times each physical activity class is assigned to a signal intensity value within one epoch. Accordingly, the physical activity duration may be determined by multiplying the count of the respective physical activity class with the duration of one measurement period.

For example, the sensor signal may be recorded regularly (e.g., once per second) and assigned to a physical activity class based on its amplitude for a specified period of time, i.e., measurement period. The classification result for each epoch may be a histogram indicating the ratio of each physical activity duration to a total duration, wherein the total duration may be the sum of all physical activity durations of the epoch and/or may be the duration of the epoch itself. The histograms of different epochs may then be used to evaluate a change in the patient's activity, i.e., to determine the pain score.

According to an embodiment, determining the behavior assessment score may comprise: determining a physical activity trend for each physical activity class from the physical activity durations of the physical activity class in at least two consecutive epochs; determining the behavior assessment score by comparing the physical activity trends. The physical activity trends may be compared with each other and/or with one or more baselines to determine the behavior assessment score and/or a behavior assessment score trend.

For example, the baseline may be a reference activity trend which may be associated with a corresponding reference behavior assessment score trend.

The baseline may have been determined with respect to the same patient, a different patient or a group of patients.

The baseline may be static. Alternatively, as mentioned above, the base line may be modified automatically or manually, e.g., based on the pain score, on a behavior assessment score trend over different epochs, on significant changes in the sensor signal or on an input of the patient.

This may significantly improve the accuracy of the method.

According to an embodiment, the signal intensity value may be determined by integrating the sensor signal over the measurement period. For example, the sensor signal may be generated in each measurement period in a sequence of time steps. Accordingly, the sensor signals of different time steps may be integrated over the respective measurement period. The sensor signal may be filtered before and/or after integration. Moreover, the sensor signal may be integrated several times, e.g., before and/or after filtering.

According to an embodiment, the method may further comprise: determining a frequency spectrum of the sensor signal in each measurement period; analyzing the frequency spectrums to detect a physical activity of the patient. Accordingly, the behavior assessment score may be determined additionally in dependence of the detected physical activities of different epochs.

For example, the frequency spectrums may be analyzed in dependence of the (previously obtained) classification results, i.e., in dependence of the physical activity classes determined from the signal intensity values.

It is possible that the frequency spectrums are analyzed only in specific frequency bands which may be relevant for detecting specific types of body movements indicative of a change in the patient's quality of life, such as, for example, (pain-related) posture changes or respiration movements.

For example, the frequency spectrums may be analyzed to detect a shift of the signal power distribution across frequencies. Such a shift may be correlated with changes in the patient's physical activity and, thus, changes in the patient's pain state, i.e., quality of life.

Optionally, the frequency spectrums may be classified with different physical activity classes (which may be the same as and/or may differ from those used to classify the signal intensity values) to obtain an additional classification result for each epoch. Accordingly, the behavior assessment score may be determined additionally from the additional classification results of different epochs.

This may significantly improve the accuracy of the method.

According to an embodiment, the sensor signal may indicate at least one of an acceleration, angular rate, translational velocity, angular velocity, position or orientation with respect to the patient, i.e., to one, two or three axes of a three-dimensional space in which the patient is moving. In other words, the sensor signal may have been generated by at least one of an accelerometer or a gyroscope. Such a sensor can provide accurate data relevant for pain assessment and is relatively inexpensive.

According to an embodiment, the sensor signal may have been generated by a sensor worn by the patient. This helps to simplify the pain monitoring over longer periods of time.

According to an embodiment, the sensor signal may have been generated by a sensor implanted in the patient's body. For example, the sensor may be part of an implantable pulse generator of a neurostimulator such as a spinal cord stimulator.

It has to be noted that possible features and advantages of embodiments of the invention are described above and below partly with reference to a method for assessing pain, partly with reference to a corresponding data processing device. A person skilled in the art will recognize that the features described for individual embodiments can be transferred, adapted and/or interchanged in an analogous and suitable manner to other embodiments in order to arrive at further embodiments of the invention and possibly synergistic effects.

Advantageous embodiments of the invention are further explained below with reference to the accompanying drawings. Neither the drawings nor the description is to be interpreted as limiting the invention.

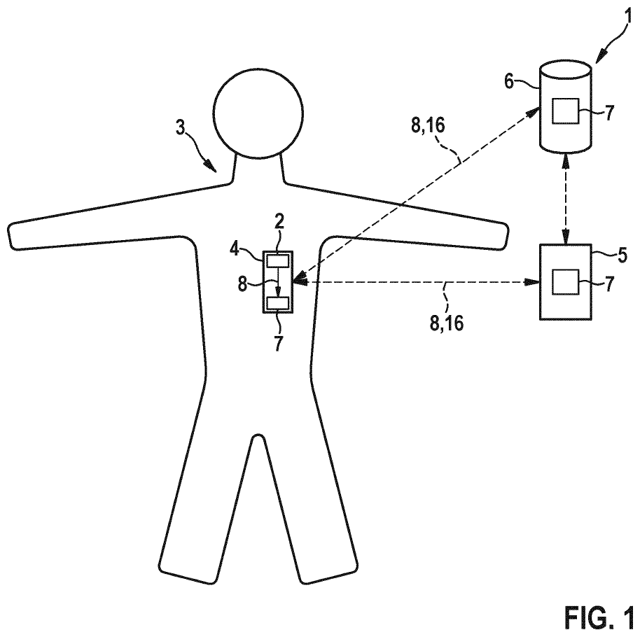

FIG. 1 shows a medical system according to an embodiment of the invention.

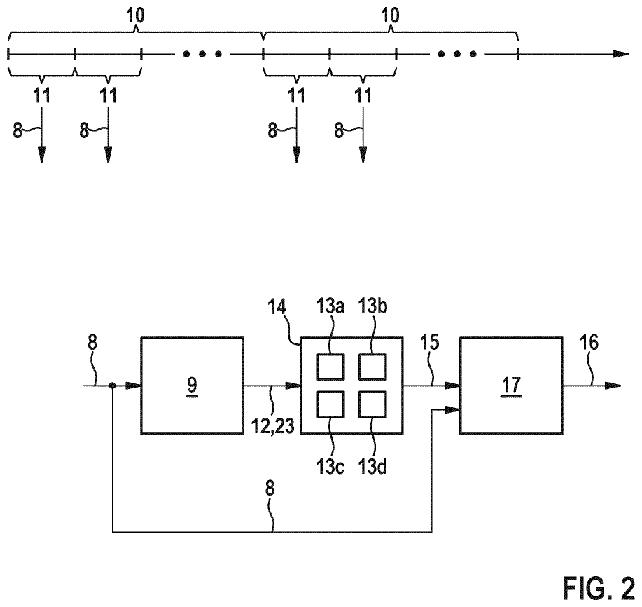

FIG. 2 shows modules of a data processing device according to an embodiment of the invention.

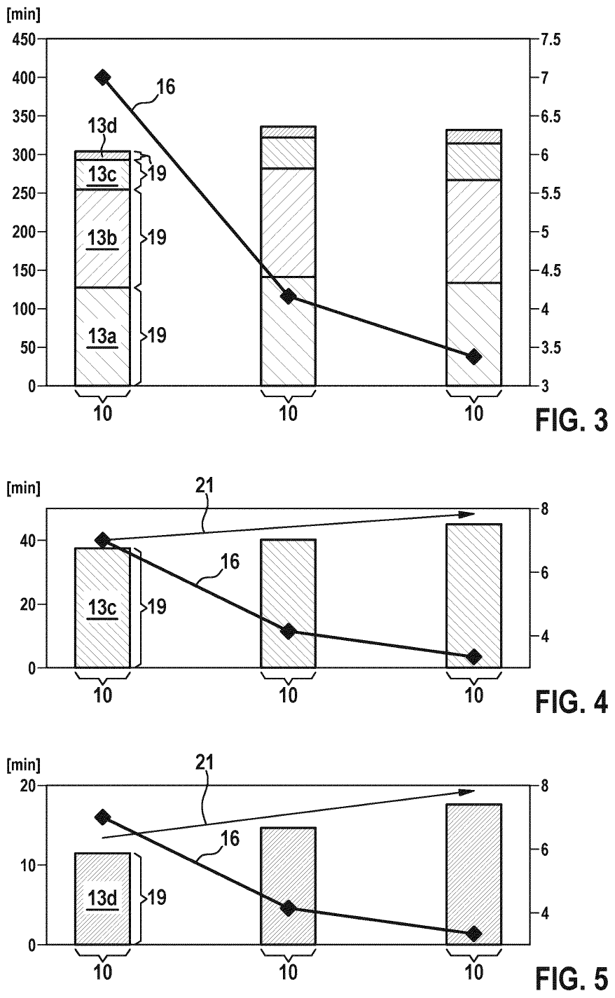

FIG. 3 shows histograms generated in a method according to an embodiment of the invention over three consecutive epochs and corresponding pain scores.

FIG. 4 shows a diagram illustrating durations of moderate physical activity over three consecutive epochs and corresponding pain scores.

FIG. 5 shows a diagram illustrating durations of vigorous physical activity over three consecutive epochs and corresponding pain scores.

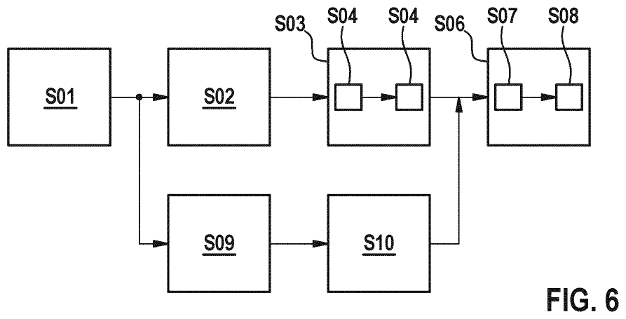

FIG. 6 shows a flow chart illustrating a method according to an embodiment of the invention.

FIG. 1 shows a medical system 1 which comprises a sensor 2 configured for measuring a physical activity of a patient 3.

The sensor 2 may be part of an implant 4, such as a neurostimulator, which is implanted in the body of the patient 3.

For example, the sensor 2 may be an accelerometer and/or a gyroscope. Other types of sensors are possible as well (see above).

The medical system 1 may further comprise a mobile device 5, e.g., a remote, smartphone, smartwatch or tablet. The mobile device 5 may be configured for controlling the implant 4.

Alternatively, the sensor 2 may be part of the mobile device 5 or part of both the implant 4 and the mobile device 5.

Additionally, the medical system 1 may include a server 6. The implant 4 and the mobile device 5 may be interconnected for data communication. Furthermore, the mobile device 5 and the server 6 may be interconnected for data communication. In addition, the implant 4 and the server 6 may be interconnected for data communication. Data communication links between these devices may be wired or wireless. The data communication may take place at least partially via the Internet.

At least one of the implant 4, the mobile device 5 or the server 6 may be equipped with a processor 7 configured for executing a computer program which causes the processor 7 to carry out a method for assessing pain experienced by the patient 3, as described in the following. The modules as described below may be software and/or hardware modules.

In step S01 (see FIG. 6), a sensor signal 8 is received in a first module 9 (see FIG. 2). The sensor signal 8 has been generated in a sequence of epochs 10 (see FIG. 2) by the sensor 2. Each epoch 10 comprises a sequence of measurement periods 11 in which the sensor signal 8 has been generated.

In step S02, a signal intensity value 12 is determined by the first module 9 for each measurement period 11 from the sensor signal 8.

The signal intensity value 12 may be determined by integrating the sensor signal 8 over the respective measurement period 11. Alternatively, the signal intensity value 12 may be a statistical value such as a mean or median. The statistical value may have been derived from the sensor signal 8 before and/or after integration.

In step S03, the signal intensity values 12 of each epoch 10 are classified with different physical activity classes 13a, 13b, 13c, 13d by a second module 14 to obtain a classification result 15 for the respective epoch 10.

For example, the physical activity classes 13a, 13b, 13c, 13d may comprise at least a first physical activity class 13a corresponding to sedentary activities, a second physical activity class 13b corresponding to light activities, a third physical activity class 13c corresponding to moderate activities and a fourth physical activity class 13d corresponding to vigorous activities.

A sedentary activity may be an activity corresponding to 1.5 MET or lower, e.g., sitting or watching TV.

A light activity may be an activity corresponding to a range between 1.5 MET and 3.0 MET, e.g., computer work, standing or walking very slow.

A moderate activity may be an activity corresponding to a range between 3.0 MET and 6.0 MET, e.g., walking brisk, cleaning, mowing lawn or biking.

A vigorous activity may be an activity corresponding to 6.0 MET or higher, e.g., hiking, jogging or biking fast.

Alternatively, there may be only three or two different physical activity classes.

Step S03 may additionally comprise step S04 in which each signal intensity value 12 is compared to different value ranges which are each linked to one of the physical activity classes 13a, 13b, 13c, 13d. If one of the signal intensity values 12 is found to be included in one of the value ranges, the signal intensity value 12 is assigned, in step S05, to the corresponding physical activity classes 13a, 13b, 13c, 13d.

In step S06, a behavior assessment score 16 indicative of the pain experienced by the patient 3 is determined from the classification results 15 of different epochs 10 by a third module 17.

It may be that the behavior assessment score 16 is determined by the third module 17 by further analyzing the sensor signal 8, e.g., its amplitude and/or frequency, based on at least two of the (previously obtained) classification results 15.

For example, the classification in step S03 may be done in such a way that the classification result 15 of each epoch 10 indicates a physical activity duration 19 (see FIG. 3, FIG. 4 and FIG. 5), here in minutes (see left vertical axis), with respect to each physical activity class 13a, 13b, 13c, 13d of the same epoch 10 (the right vertical axis in FIG. 2, FIG. 4 and FIG. 5 indicates the amount of the behavior assessment score 16).

In this case, step S06 may additionally comprise step S07 in which a physical activity trend 21 for each physical activity class 13a, 13b, 13c, 13d is determined by the third module 17 from the physical activity durations 19 of the respective physical activity class in at least two consecutive epochs 10.

By way of example, FIG. 4 shows the physical activity trend 21 relating to the third physical activity class 13c, i.e., moderate activity, whereas FIG. 5 shows the physical activity trend 21 relating to the fourth physical activity class 13d, i.e., vigorous activity. Both physical activity trends 21 relate to the same sequence of three consecutive epochs 10.

Accordingly, the third module 17 may determine the behavior assessment score 16 by comparing, in step S08, the physical activity trends 21 of the different physical activity classes 13a, 13b, 13c, 13d with respect to the same sequence of epochs 10.

In additional step S09, a frequency spectrum 23 of the sensor signal 8 may be determined in each measurement period 11 by the first module 9.

In additional step S10, the frequency spectrums 23 may be analyzed by the second module 14 to detect a physical activity of the patient 3 over at least two epochs 10. The two epochs 10 may be two consecutive epochs.

The results of step S10 may be used in addition to the classification results 15 by the third module 17 to determine the behavior assessment score 16 in step S06, e.g., by further analyzing the sensor signal 8.

It is possible that the behavior assessment score 16 is determined by the implant 4 itself and transmitted from the implant 4 to the mobile device 5 and/or to the server 6 for further processing.

Another possibility is that the sensor signal 8 is transmitted from the implant 4 to the mobile device 5 and/or from the implant 4 directly to the server 6 and/or from the implant 4 via the mobile device 5 to the server 6. Accordingly, the behavior assessment score 16 may be determined from the sensor signal 8 by the mobile device 5 and/or by the server 6.

The behavior assessment score 16 may be used by the mobile device 5 and/or the server 6 to control the implant 4, e.g., a pulse generator of the implant 4.

For example, the sensor signal 8 may indicate motion data which may be analyzed in different intensity ranges.

In this case, the method may comprise the following steps:
1) Calculate the intensity of the sensor signal 8, i.e., integrate the sensor signal 8 over a certain (relatively) short measurement period 11, e.g., 1 second or 15 seconds, to generate a signal intensity value 12.
2) Evaluate the signal intensity values 12 according to multiple physical activity classes 13a, 13b, 13c, 13d, e.g., multiple levels of activity, and increment a counter of the respective physical activity class by one.
3) Define the duration of one epoch 10, e.g., 10 minutes or 24 hours, over which the activity bins (here four bins; see FIG. 3) are accumulated to form an activity histogram for that epoch 10, and store these values representing that epoch to memory, also clearing the four activity bins for the next epoch 10.
4) Monitor the trend of the activity intensity distribution over multiple epochs 10, and interpret the change of the activity levels of interest as being indicative of the change of the patient's ability to move around, work or exercise, and provide information on the life quality improvement. For instance, increased “high-intensity” motion signals 8 may indicate that the patient 3 suffers less from the pain and is able to integrate more movement in their daily life.

The change can, for example, refer to the duration of the activity signals, the average and/or median intensity of the activity, the consistency of activity signals or the relative distribution of time spent in each activity range over a day.

As mentioned above, the sensor signal 8 may also be analyzed in different frequency ranges. In this case, the signal power of the sensor signal 8 may be additionally calculated within a specified frequency bandwidth.

The sensor signal 8 within a certain frequency range can be used to analyze and characterize the change of the patient's specific type of movement. For instance, DC signals can indicate the posture changes and duration each day. If the patient 3 used to be unable to get off the bed due to the pain before treatment and experiences less pain after the treatment, the DC signals can detect this change.

The sensor signals 8 at different frequency bands can indicate the specific activities such as respiration, exercise, etc.

As the quality of life of the patient 3 improves, the signal power distribution across frequencies may shift significantly. For instance, walking faster or moving around with less wariness about pain may produce a higher proportion of high-frequency motion signals 8.

As can be seen from FIG. 3, the behavior assessment score 16 (here on the Numeric Rating Scale) decreases as the physical activity durations 19 increases, especially with respect to the third and fourth physical activity classes 13c, 13d, i.e., moderate and vigorous activity. In detail, in the left epoch 10, the behavior assessment score 16 is around 7 and the patient 3 is in moderate activity for about 38 minutes, and in vigorous activity for about 12 minutes. In the right epoch 10, the behavior assessment score 16 is around 3.3 and the durations 19 of both moderate and vigorous activity are significantly higher than in the left epoch 10 (see also FIG. 4 and FIG. 5).

It has to be noted that, in the claims, the word “comprising” does not exclude other elements or steps, and the indefinite article “a” or “an” does not exclude a plurality. A single processor or controller or other unit may fulfil the functions of several items recited in the claims. The mere fact that certain measures are recited in mutually different dependent claims does not indicate that a combination of these measures cannot be used to advantage. Any reference signs in the claims should not be construed as limiting the scope of the claims.

1. A computer-implemented method for assessing behavior associated with a medical condition of a patient (3), the method comprising:
receiving a sensor signal (8) which has been generated in a sequence of epochs (10) by a sensor (2) configured for measuring a physical activity of the patient (3), wherein each epoch (10) comprises a sequence of measurement periods (11), wherein the sensor signal (8) has been generated in each measurement period (11);
determining a signal intensity value (12) for each measurement period (11) from the sensor signal (8); and
classifying the signal intensity values (12) of each epoch (10) with different physical activity classes (13a, 13b, 13c, 13d) to obtain a classification result (15) for the epoch (10).

2. The method of claim 16,
wherein the behavior assessment score (16) is determined by further analyzing the sensor signal (8) in dependence of the classification results (15) of different epochs (10) by collecting the classification results within an epoch to determine the amount of activity of each activity class within that epoch.

3. The method of claim 1,
wherein classifying the signal intensity values (12) comprises:
comparing each signal intensity value (12) to different value ranges, each value range corresponding to one of the physical activity classes (13a, 13b, 13c, 13d);
when a value range which includes the signal intensity value (12) is found: assigning the physical activity class (13a, 13b, 13c, 13d) of the found value range to the signal intensity value (12).

4. The method of claim 1,
wherein the physical activity classes (13a, 13b, 13c, 13d) include at least one of a first physical activity class (13a) comprising sedentary activities, a second physical activity class (13b) comprising light activities, a third physical activity class (13c) comprising moderate activities, or a fourth physical activity class (13d) comprising vigorous activities.

5. The method of claim 1,
wherein the classification result (15) indicates a physical activity duration (19) with respect to each physical activity class (13a, 13b, 13c, 13d); and
wherein the behavior assessment score (16) is determined from the physical activity durations (19) of different epochs (10).

6. The method of claim 5,
wherein determining the behavior assessment score (16) comprises:
determining a physical activity trend (21) for each physical activity class (13a, 13b, 13c, 13d) from the physical activity durations (19) of the physical activity class (13a, 13b, 13c, 13d) in at least two consecutive epochs (10); and
determining the behavior assessment score (16) by comparing the physical activity trends (21).

7. The method of claim 1,
wherein the signal intensity value (12) is determined by integrating the sensor signal (8) over the measurement period (11).

8. The method of claim 1, further comprising:
determining a frequency spectrum (23) of the sensor signal (8) in each measurement period (11); and
analyzing the frequency spectrums (23) to detect a physical activity of the patient (3);
wherein the behavior assessment score (16) is determined additionally in dependence of the detected physical activities of different epochs (10).

9. The method of claim 1,
wherein the sensor signal (8) indicates at least one of an acceleration, angular rate, translational velocity, angular velocity, position, or orientation with respect to the patient (3).

10. The method of claim 1,
wherein the sensor signal (8) has been generated by a sensor (2) worn by the patient (3).

11. The method of claim 10,
wherein the sensor signal (8) has been generated by a sensor (2) implanted a body of the patient.

12. A data processing device comprising a processor (7) configured for carrying out the method of claim 1.

13. A medical system (1), comprising:
at least one sensor (2) configured for measuring a physical activity of a patient (3); and
at least one data processing device (4, 5, 6) according to claim 12.

14. A computer program comprising instructions which, when the program is executed by a processor (7), cause the processor (7) to carry out the method of claim 1.

15. A computer-readable medium comprising instructions which, when executed by a processor (7), cause the processor (7) to carry out the method of claim 1.

16. The method of claim 1,
further comprising determining a behavior assessment score (16) indicative of the pain experienced by the patient (3) from the classification results (15) of different epochs (10).

17. The medical system of claim 13, further comprising:
an implant (4);
a mobile device (5); and
a server (6);
wherein the implant is configured to transmit the sensor signal (8) to one or both of the mobile device (5) or the server (6).

18. The medical system of claim 17, wherein the implant is configured to transmit the sensor signal (8) to the server (6) via the mobile device (5).
